(19) United States
(12) Patent Application Publication
(10) Pub. No.: US 2026/0224779 A1
(43) Pub. Date: Aug. 6, 2026

(54) FELTED IMPLANT AND METHOD OF IMPLANTATION

(71) Applicants: Smith & Nephew, Inc., Memphis, TN (US); Smith & Nephew Asia Pacific Pte. Limited, Singapore (SG); Smith & Nephew Orthopaedics AG, Zug (CH)

(72) Inventors: Justin A. Callaway, Goffstown, NH (US); Timothy Young, Upton, MA (US); Nehal N. Patel, Boston, MA (US)

(73) Assignees: Smith & Nephew, Inc., Memphis, TN (US); Smith & Nephew Asia Pacific Pte. Limited, Singapore (SG); Smith & Nephew Orthopaedics AG, Zug (CH)

(21) Appl. No.: 19/576,370

(22) Filed: Mar. 24, 2026

Related U.S. Application Data
(63) Continuation of application No. PCT/US2024/049712, filed on Oct. 3, 2024.
(60) Provisional application No. 63/542,855, filed on Oct. 6, 2023.

(51) Int. Cl. A61L 27/24; A61L 27/54; A61L 27/58

(52) U.S. Cl. CPC A61L 27/24; A61L 27/54; A61L 27/58; A61L 2430/34

(57) ABSTRACT

A tissue repair implant includes a sheet-like structure having a first component comprising a biological material, and a second component comprising a fibrous felt material. The second component is configured to pass entirely through a thickness of the first component to extend into and engage with a body tissue to affix the repair implant to the tissue.

### CROSS REFERENCE TO RELATED APPLICATIONS

This application is a continuation of International Application Serial No. PCT/US2024/049712, filed on October 3, 2024, which claims the benefit of U.S. Patent Application Serial No. 63/542,855 filed on October 6, 2023, the disclosures of which are incorporated herein by reference.

### TECHNICAL FIELD

The present disclosure pertains generally, but not by way of limitation, to orthopedic implants and methods of treatment. More particularly, the present disclosure relates to a felted tendon repair implant, such as one that is engineered for placement in the area of a tear or lesion of a tendon or ligament, and methods for promoting and enhancing healing at a soft tissue-to-bone interface in a surgical repair.

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear, or from overuse of the joint. In repairing tendon tears, including partial thickness tears and full thickness tears, a bioinductive implant may be used to induce new tendinous tissue, which biologically augments the tendon and enables healing of tendon defects. The healing response associated the bioinductive implant results in integration of the newly induced tissue with the native tissues, which provides a biological connection for tissue regeneration and load sharing during the healing process. There is an ongoing need to deliver and adequately position medical implants during an arthroscopic procedure in order to treat injuries to the rotator cuff, rotator cuff tendons, or other soft tissue or tendon injuries throughout a body.

### BRIEF SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

An example medical device may include a tissue repair implant including a sheet-like scaffold having a first component and a second component. The first component may include a biological material and the second component may include a fibrous felt material formed of a plurality of fibers, wherein the fibers of the second component engage with the first component.

Alternatively or additionally to any of the embodiments above, the fibers of the second component may extend entirely through a thickness of the first component to engage with a tissue to affix the tissue repair implant to the tissue.

Alternatively or additionally to any of the embodiments above, the fibers of the second component may be glued to the first component

Alternatively or additionally to any of the embodiments above, the fibers of the second component are pre-felted into the first component.

Alternatively or additionally to any of the embodiments above, the biological material may be a collagen layer and the fibrous felt material may be a felt layer juxtaposed with an upper surface of the collagen layer.

Alternatively or additionally to any of the embodiments above, the felt material may be a resorbable felt.

Alternatively or additionally to any of the embodiments above, the biological material may be collagen.

Alternatively or additionally to any of the embodiments above, the second component extends across only a portion of an upper surface of the first component.

Alternatively or additionally to any of the embodiments above, the second component is secured to the first component only around an outer perimeter of the sheet-like scaffold.

Alternatively or additionally to any of the embodiments above, the fibers of the second component are intermingled within the first component via felting.

Alternatively or additionally to any of the embodiments above, fibers of the second component includes a color that differs from a color of the first component.

Another example tissue repair implant may include a sheet-like scaffold including a first layer having an absorbable material, and a second layer having a fibrous felt material formed of a plurality of fibers. The second layer may be disposed along and juxtaposed with an upper surface of the first layer to secure the second layer to the first layer, and the fibers of the felt material may extend into the first layer to secure the second layer to the first layer.

Alternatively or additionally to any of the embodiments above, the absorbable material may be collagen having collagen fibers.

Alternatively or additionally to any of the embodiments above, the fibers of the felt material may be intertwined with collagen fibers.

Alternatively or additionally to any of the embodiments above, the felt material may be a resorbable felt.

Alternatively or additionally to any of the embodiments above, the felt material may be a non-resorbable felt.

Alternatively or additionally to any of the embodiments above, the fibers of the second layer may include a color that differs from a color of the first layer.

Alternatively or additionally to any of the embodiments above, the fibers of the felt material may extend entirely through a thickness of the first layer to engage with a tissue to affix the tissue repair implant to the tissue.

An example method of securing a tissue repair implant to a damaged tissue may include placing a sheet-like implant over the damaged tissue. The sheet- like implant may include a first component having a biological material, and a second component having a fibrous felt material formed of a plurality of fibers. The method may further include securing the sheet-like implant over the damaged tissue by felting the fibers of the second component through the first component to engage with the damaged tissue to affix the tissue repair implant to the tissue.

Alternatively or additionally to any of the embodiments above, the second component may be attached to the first component prior to placing the sheet-like implant over the damaged tissue.

Alternatively or additionally to any of the embodiments above, fibers of the felt material may be intertwined with collagen fibers of the biological material.

Alternatively or additionally to any of the embodiments above, the fibers of the second component may be intermingled within the first component around an outer perimeter of the sheet-like implant.

Alternatively or additionally to any of the embodiments above, the step of placing the sheet-like implant over the damaged tissue may include placing the first component against the damaged tissue, and thereafter, placing the second component against an upper surface of the first component only after the first component is placed against the damaged tissue.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is an enlarged schematic cross-sectional view of a shoulder showing a partial thickness tear of a tendon;

FIG. 2A is a schematic representation of an exemplary tendon repair scaffold, including a felting fiber component;

FIG. 2B is a schematic representation of an exemplary tendon repair scaffold, including felting fibers of a felting fiber component extending through a biological material component of the scaffold;

FIG. 3 is an enlarged schematic cross-sectional view of a shoulder showing a partial thickness tear and an exemplary tendon repair scaffold positioned thereon;

FIG. 4 is a schematic representation of an exemplary tendon repair scaffold, showing a felting fiber component only at a first end of the tendon repair scaffold;

FIG. 5 is a schematic representation of an exemplary tendon repair scaffold, showing a felting fiber component only at a second end of the tendon repair scaffold;

FIG. 6 is an enlarged perspective view of a shoulder showing an exemplary tendon repair scaffold positioned over a tendon tear;

FIG. 6A is a cross-sectional view taken along line 6A-6A of FIG. 6;

FIG. 7 in a schematic representation of an exemplary tendon repair scaffold including a fibrous felting layer; and

FIG. 8 is a schematic cross-sectional view of the exemplary tendon repair scaffold shown in FIG. 6, taken along line 8-8.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification.

All numeric values are herein assumed to be modified by the term "about," whether or not explicitly indicated. The term "about" generally refers to a range of numbers that one of skill in the art would consider equivalent to the recited value (i.e., having the same function or result). In many instances, the term "about" may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms "a", "an", and "the" include plural referents unless the content clearly dictates otherwise. As used in this specification and the appended claims, the term "or" is generally employed in its sense including "and/or" unless the content clearly dictates otherwise.

It is noted that references in this specification to "an embodiment", "some embodiments", "other embodiments", etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the claims.

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear, or from overuse of the joint. Current repair procedures may attempt to alleviate impingement or make room for movement of the tendon to prevent further damage and relieve discomfort. An accepted treatment for rotator cuff tears may include reattaching the torn tendon to the humeral head using sutures. Additionally, in treating rotator cuff tears, an accepted practicemay also include the placement of a biocompatible scaffold or implant over the repaired tendon to mechanically reinforce the repaired tendon. Therefore, there is an ongoing need to deliver and adequately position medical implants during an arthroscopic procedure in order to treat injuries to the rotator cuff, rotator cuff tendons, or other soft tissue or tendon injuries throughout a body.

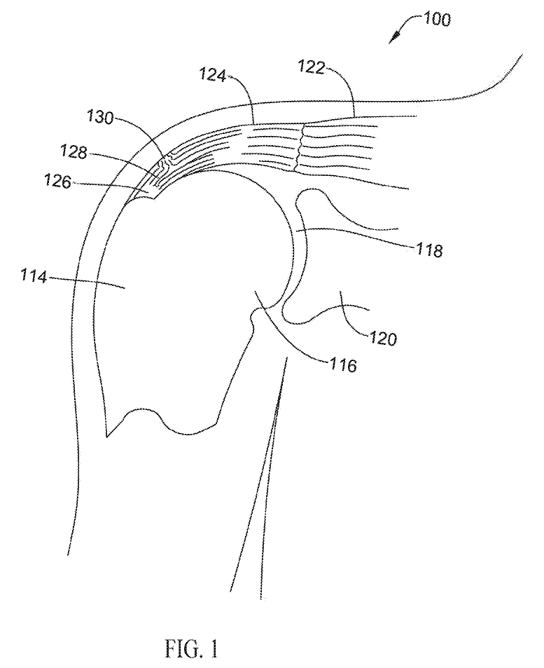

FIG. 1 is an enlarged schematic cross-sectional view of a shoulder 100 showing a tendon tear 130, shown as a partial thickness tear. The shoulder 100 further shows a head 114 of a humerus 116 mating with a glenoid fossa 118 of scapula 120. The glenoid fossa 118 includes a shallow depression in scapula 120. A supraspinatus tendon 122 is also shown connected to the supraspinatus muscle of the rotator cuff. These muscles (along with others) control the movement of the humerus 116 relative to the scapula 20. A distal tendon 124 of the supraspinatus tendon 122 meets the humerus 116 at an insertion point 126.

In FIG. 1, the distal tendon 124 includes a damaged portion 128 located near the insertion point 126. The damaged portion 128 includes a tear 130 extending partially through the distal tendon 124. The tear 130 may be referred to as a partial thickness tear. The depicted partial thickness tear 130 is on the bursal side of the tendon, however, the tear may also be on the opposite or articular side of the distal tendon 124 and/or may include internal tears to the distal tendon 124 not visible on either surface. In other instances, the tear 130 may be a full thickness tear. As will be discussed herein, an implant (not explicitly shown in FIG. 1) may be used to repair the tear 130.

Although one possible treatment site is described in the figures as being located in a shoulder joint, an implant may be used at a variety of different treatment sites, such as the hip, knee, ankle, etc. Furthermore, an implant may be used for any of a variety of soft tissue repairs, such as but not limited to the Gluteus Medius, which is a large fan-shaped muscle located in the posterior hip, the Hip Capsule, which is also in the hip. The implant may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The implant may be used in treating soft tissue injuries in the Achilles tendon. The implant may be used in treating soft tissue injuries for any areas of the body that will accommodate the implant.

In some instances, delivery of an implant (e.g., a sheet-like implant) to a target site of a patient may require a physician to create an incision in the patient sufficient to access the target implant site. After creating this "access site," the physician may insert an implant delivery system through the access site and position the distal end of the implant delivery system adjacent the target implant site. The physician may then manipulate the implant delivery system to deploy the implant adjacent the target implant site.

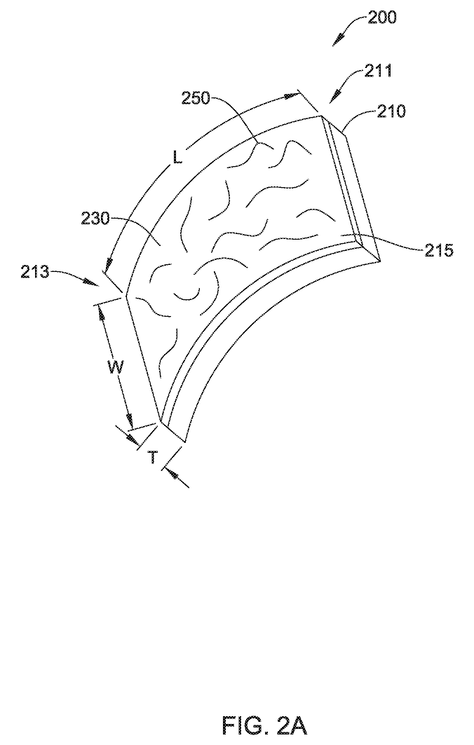

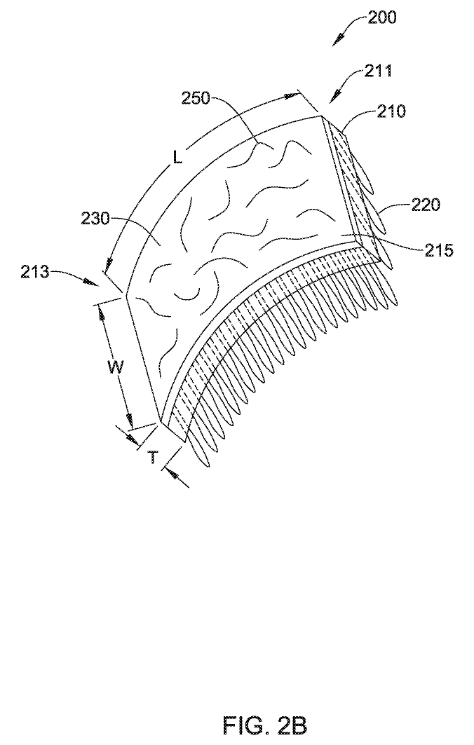

FIG. 2A is a schematic representation of an exemplary tendon repair implant 200. As shown in FIG. 2A, the repair implant 200 may comprise a sheet-like structure having a first, medial edge 211 and a second, lateral edge 213, opposite the first edge 211. The repair implant 200 may include a first component 210 comprising a biological material (e.g., collagen) and a second component 230 comprising a fibrous material (e.g., resorbable felt, a non-absorbable felt, a biological material) including a plurality of fibers 250. In some cases, the fibers 250 of the second component 230 extend into and/or may be interwoven within the first component 210, and may extend through the repair implant 200 to engage with a tissue (e.g., the distal tendon 124) to affix the repair implant 200 to the tissue, as will be discussed further herein. In some cases, the first component 210 and the second component 230 may not be woven together, but rather the first component 210 may be joined to the second component 230 via any other suitable manner, such as for example, pre-felting fibers 250 of the second component 230 into the first component 210, glue, sutures, or the like. In some cases, the first component 210 and the second component 230 are separate distinct layers that may be integrated with one another either in a homogeneous fashion or a heterogeneous fashion. For example, the first component 210 and the second component 230 may be arranged one on top of the other with a surface (e.g., upper surface) of the first component 210 facing and juxtaposed with a surface (e.g., lower surface) of the second component 230. For example, as shown in FIG. 2A, the second component 230 may be placed on top of the first component 210. In some cases, the second component 230 may be positioned on top of the first component 210 and connected to the first component 210, such as including fibers of the second component 230 extending into and/or interwoven within the first component 210 around an outer perimeter 215 of the repair implant 200. In other words, in some embodiments, fibers of the second component 230 may extend into and/or through the first component 210 only around the periphery of the repair implant 200, while the central portion of the repair implant 200 is devoid of fibers of the second component extending into and/or through the first component 210. In some cases, the second component 230 may be positioned on top of the first component 210 with fibers 250 extending into and/or interwoven within or through only a portion of the first component 210, such as a medial region of the first component 210 proximate the medial edge 211 of the first component 210 and/or a lateral region of the first component 210 proximate the lateral edge 213. In other words, in some embodiments, fibers of the second component 230 may extend into and/or through the first component 210 only along the medial region of the repair implant 200, while the lateral region of the repair implant 200 is devoid of fibers of the second component extending into and/or through the first component 210, or fibers of the second component 230 may extend into and/or through the first component 210 only along the lateral region of the repair implant 200, while the medial region of the repair implant 200 is devoid of fibers of the second component extending into and/or through the first component 210. Other configurations of the arrangement of the second component 230 with the first component 210 are also contemplated. In some cases, the second component 230 may be positioned on top of the first component 210 with fibers of the second component 230 extending int and/or interwoven within the entirety of the first component 210. In some instances, the second component 230 may be coterminous with the first component 210 such that the edges of the second component 230 are aligned with the edges of the first component 210 and covers an entire extent of the upper surface of the first component 210. In other instances, the second component 230 may be of a different size and/or shape than the first component 210. For example, the second component 230 may be smaller than the first component 210 such that the second component 230 only covers a portion of the upper surface of the first component 210. The second component 230 may be placed along a periphery of the first component 210, with an upper surface of the first component 210 exposed throughout a central portion of the repair implant 200, placed along the medial edge of the first component 210 with an upper surface of the first component 210 exposed along a lateral edge of the repair implant 200, placed along a lateral edge of the first component 210 with an upper surface of the first component 210 exposed along a medial edge of the repair implant 200, extend along a central region of the first component 210 with an upper surface of the first component 210 exposed along both the medial and lateral edges of the repair implant 200, or otherwise positioned over only a portion of the first component 210.

In other cases, the second component 230 may be distributed throughout the first component 210, without distinct separate "layers". For example, on a semi- continuous basis such as not to form distinct layers, a fibrous material (of the second component 230) could be intermingled with a biological material of the first component 210) in order to produce a woven or non-woven composite structure of the first and second components 210, 230.

In some cases, the second component 230 may be formed from a material that is amenable to a felting process. For example, the second component 230 may be a resorbable felt or a non-absorbable felt including a plurality of felting fibers 250 or strands. In some cases, the second component 230 may be a biological material, such as an absorbable material. In some cases, the second component 230 includes a plurality of fibers 250 forming a fabric. In some cases, the second component 230 may be formed from collagen, polyethylene, UHMWPE, PLA, PLLA, PGA, PLDL, PET, nylon, ABS, or any other suitable material. In some cases, the second component 230 may include a color that differs from a color of the first component 210 to distinguish the placement, location, arrangement, etc. of the felting fibers 250 of the second component 230 of the implant 200. For example, the second component 230 may be blue, green, yellow, red, or any other suitable color.

In some embodiments, the tendon repair implant 200 may include a selected porosity and/or longitudinal pathways for tissue in-growth. In some embodiments, the first component 210 of the implant 200 may comprise a materialdefining a plurality of pores that encourage tissue growth therein. In some cases, the first component 210 may be a non-woven fibrous construct, such as a collagen-based scaffold. In some instances, the first component 210 may be a collagen construct having longitudinally oriented collagen filaments. In other instances, the first component 210 may be a woven construction including a weave pattern, for example. In some cases, a coating or substance that encourages tissue growth or in-growth may be applied to the surfaces of the repair implant 200. The porosity and tissue in- growth allow for new collagen to integrate with collagen of the native tendon for biological healing. The felting process, which may affix the tendon repair implant 200 to the tissue (e.g., distal tendon 124), may be distributed across various regions of the tendon repair implant 200 and the tissue interface. This distribution results in functional load carrying. Further, the porosity of the first component 210 of the implant 200 may be configured to allow fibers 250 of the second component 230 to be felted therethrough. For example, when fibers 250 of the second component 230 are felted through the thickness of the first component 210 of the implant 200, the pore size at an upper surface of the first component 210 may be larger than that of the pore size at a lower surface of the first component 210 of the implant 200. In such cases, fibers 250 of the second component 230 may extend through the plurality of pores of the first component 210 and form one or more loops 220 (shown in FIG. 2B) configured to secure the repair implant (sheet-like structure) 200 to the tissue. In some cases, the fibers 250 of the second component 230 may be pushed through the first component 210 as individual strands which may be configured to secure the repair implant 200 to the tissue. It will be appreciated that sheet-like structure may comprise various pore defining structures without deviating from the spirit and scope of the present description.

In some embodiments, the first component 210 of the repair implant 200 may include a pore size in the range of about 20 to about 400 microns. In some embodiments the pore size may be in the range of about 100 microns to about 300 microns, and in some embodiments, the pore size may be about 150 to about 200 microns. The porosity may be about 30% to about 90%, or it may be within the range of at least about 50% to about 80%, in some instances. Examples of pore defining structures may include, but are not limited to open cell foam structures, mesh structures, micromachined layered structures, 3-D printed structures, and structures comprising a plurality of woven or non-woven fibers. In some embodiments, the fibers may be interlinked with one another. Various processes may be used to interlink the fibers with one another. Examples of processes that may be suitable in some applications include weaving, knitting, and braiding. In other instances, the fibers 250 may be randomly arranged within the first component 210.

In some embodiments, the tendon repair implant 200 may be bioresorbable, biodegradable, bioinductive, or otherwise absorbable to provide transfer of additional load to native tendon over time. In some instances, by 2-3 months after implantation, the new tissue in-growth will have gained strength through remodeling such that more load may be transferred from the implant 200 to the new tissue and native tendon combination. Absorption of the implant 200 enables the new tissue, in combination with the native tendon, to carry all of the load and develop optimal collagen fiber alignment over time. Further, absorption avoids potential long-term problems with particles from non-absorbable materials. It is expected that tissue within the repair implant 200 will typically be developing and organizing during the first one to three months after implantation, so load sharing with the implant 200 is desired in some embodiments. After three months the tissue will typically be remodeling. As such, the mechanical properties of portions of the implant 200 may gradually decline to zero to enable the new tissue to be subjected to load without the implant 200 bearing any appreciable amount of the load.

In some useful embodiments, the tendon repair implant 200 comprises one or more bioabsorbable materi- als. Examples of bioabsorbable materials that may be suit- able in some applications include those in the following list, which is not exhaustive: polylactide, poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen, poly(hydroxybutyrate ), polyanhydride, polyphos- phoester; poly-(amino acids), poly- (alphahydroxy acid) or related copolymer materials.

The tendon repair implant 200 may be configured to allow loading and retention of biologic growth factors. The implant 200 and/or the growth factors may be configured to controllably release the growth factors. The implant 200 may be configured to allow transmission of body fluid to remove any degradation by- products in conjunction with a potential elution profile of biologics. The implant 200 may also include platelet rich plasma, placental membrane, or umbilical tissue at the time of implantation or other biologic factor to promote healing and tissue formation.

In some instances, the tendon repair implant 200 of the present disclosure may include multiple layers or surface coatings. As implanted, the bursal side of the implant 200 can include a layer or surface that will preferably slide against tissue without adherence. The tendon side of the implant 200 may include a layer or coating that is more compatible with fixation to the tendon surface.

In FIG. 2A, the tendon repair implant 200 is defined by a longitudinal dimension L (i.e., length), measured from the medial edge 211 to the lateral edge 213, a lateral dimension W (i.e., width), measured perpendicular to the longitudinal dimension L, and a thickness T, measured perpendicular to the both the longitudinal dimension L and the lateral dimension W. The thickness T is measured from the upper surface to the lower surface of the implant 200. In some embodiments, lateral and longitudinal dimensions of the repair implant 200 may range from about 20 millimeters (mm) to 50 mm in the lateral direction W and 25 mm to 70 mm in the longitudinal direction L. The thickness T of the sheet-like structure may be about 0.5 mm to 5 mm when dehydrated. It is contemplated that the thickness T of the implant 200 may be greater, in the range of about 1 mm to 10 mm, when hydrated. Upon implantation, the longitudinal dimension L may extend generally in, or parallel to, the load bearing direction of the tendon. While it is illustrated that the repair implant 200 may include a rectangular shape, it may be contemplated that the repair implant 200 may include any shape as desired. Such as, for example, an oval shape, a hexagonal shape, a diamond shape, or any other suitable shape.

In some instances, the first component 210 may be prepared and/or provided separately from the second component 230, and then the second component 230 may be positioned above/against the first component intraoperatively. For example, the first component 210 may be placed against a target tissue site, and thereafter the second component 230 may be positioned thereon. The second component 230 may be used to secure the first component 210 to the target tissue site, such as during a felting process as described herein. In some instances, the repair implant 210 may be loaded in a delivery device which manually holds the second component against the first component 210 during delivery of and placement of the repair implant 200 at the treatment site of a tendon. Upon placement at the treatment site, the fibers of the second component 230 may be felted through the thickness of the first component 210 and into underlying soft tissue to secure the repair implant, including both the first component 210 and the second component 230, to the underlying soft tissue at the treatment site. In some instances, the second component 230 may be provided pre-attached to the first component 210 as a multi-layer construct.

FIG. 2B illustrates fibers 250 from the second component 230 extending entirely through the thickness of the first component 210, from beyond an upper surface of the first component 210 to beyond a lower surface of the first component 210. Fibers 250 of the second component 230 may extend from the lower surface of the first component 210 to be embedded into, intermesh with, or otherwise engage underlying soft tissue (e.g., tendon tissue), and in some instances form loops 220 extending from the first component 210 to be embedded into, intermesh with, or otherwise engage tissue (e.g., tendon tissue) for securement of the implant 200 thereto.

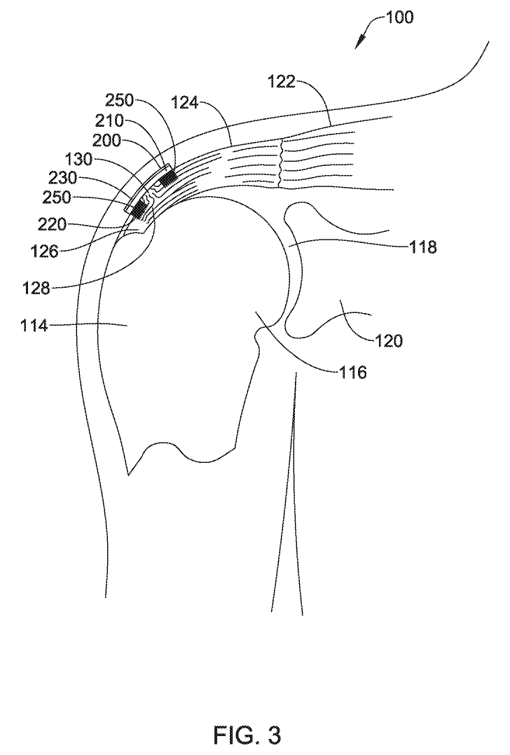

FIG. 3 is an enlarged schematic cross-sectional view of the shoulder 100 showing a tendon tear 130, and the exemplary tendon repair implant 200 positioned thereon. Similar to that shown in FIG. 1, shoulder 100 further shows a head 114 of a humerus 116 mating with a glenoid fossa 118 of scapula 120. The glenoid fossa 118 includes a shallow depression in scapula 120. A supraspinatus tendon 122 is also shown connected to the supraspinatus muscle of the rotator cuff. These muscles (along with others) control the movement of the humerus 116 relative to the scapula 20. A distal tendon 124 of the supraspinatus tendon 122 meets the humerus 116 at an insertion point 126.

In FIG. 3, the distal tendon 124 includes a damaged portion 128 located near the insertion point 126. The damaged portion 128 includes a tear 130 extending partially through the distal tendon 124. The tear 130 may be referred to as a partial thickness tear. The depicted partial thickness tear 130 is on the bursal side of the tendon, however, the tear may also be on the opposite or articular side of the distal tendon 124 and/or may include internal tears to the distal tendon 124 not visible on either surface. In other instances, the tear 130 may be a full thickness tear.

FIG. 3 further illustrates that the tendon repair implant 200 has been placed over the tear 130. In some instances, both the first component 210 and the second component 230 may be simultaneously placed across the tear 130 as a single construct, or the first component 210 and the second component 230 may be sequentially placed across the tear 130, with the first component 210 being placed directly over the tear 130 and subsequently placing the second component 230 over the first component 210.

In this example, the tendon repair implant 200 may be positioned over the tear 130 and attached to the tendon 124 via a felting process. Felting may be performed by moving a felting needle repeatedly into, and in some cases through the implant 200, including the first component 210 and the second component 230 thereby pushing fibers 250 of the felt material of the second component 230 through the entire thickness of the first component 210 and into underlying tissue, e.g., tendon tissue. Thus, fibers 250 of the second component 230 may extend through the entire thickness of the first component 210 and extend beyond the upper and lower surfaces of the first component 210. The felting needle may include barbs that catch the fibers of the felt material of the second component 230 and move the fibers 250 through the first component 210 and entangles the fibers of the felt into the underlying tissue, e.g., tendon tissue. In particular, the felting may not only entangle the fibers 250 of the felt of the second component 230 into the first component 210 that forms part of the implant 200 with each other, but also entangle the fibers 250 of the felt into the underlying tissue, e.g., tendon tissue, to secure the implant 200 thereto. Thereby, some fibers 250 of the felt become embedded in and entangled with the patient's tissue and some strands of the tissue fibers may become entangled with the felt fibers 250 extending from the lower surface of the repair implant 200. This creates a strong and well distributed mechanical bond between the implant 200 and the underlying body tissue, e.g., tendon tissue.

In the example shown in FIG. 3, a method of securing the repair implant 200 to a damaged tissue site, e.g., tendon tear 130, may include placing the sheet-like implant 200 over the damaged tissue site. The sheet-like implant 200 may include a sheet-like structure having the first component 210 comprising a biological material, and the second component 230 comprising a fibrous felting material. In some cases, the second component 230 may be secured with the first component 210 as a single construct prior to placement over the tendon tear 130. For example, fibers of the second component 230 may be intertwined within the first component 210 prior to placement over the tendon tear 130. In other instances, the second component 230 may be placed over the previously placed first component 210 intraoperatively. The implant 200 may be secured over the damaged tissue by felting the second component 230 through the first component 210 to engage fibers of the second component 230 (e.g., felting fibers) with the damaged tissue to affix the repair implant 200 to the tissue. In some cases, the second component 230 extends through the plurality of pores of the first component 210 and forms one or more loops 220 configured to attach the implant 200 to the tissue.

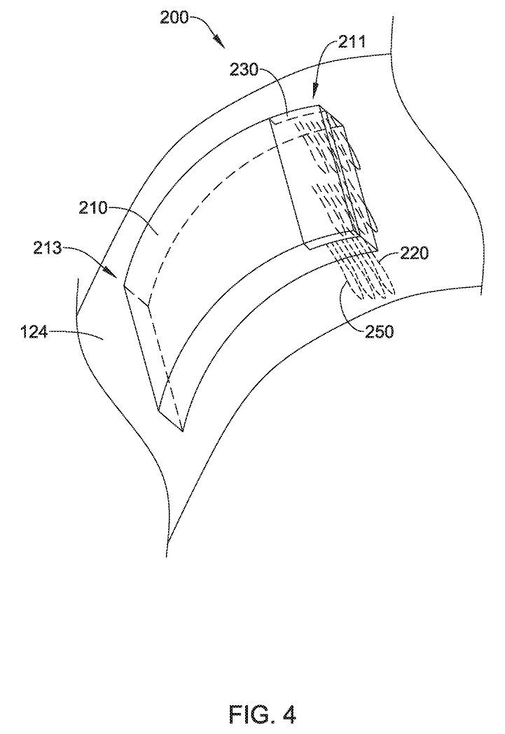

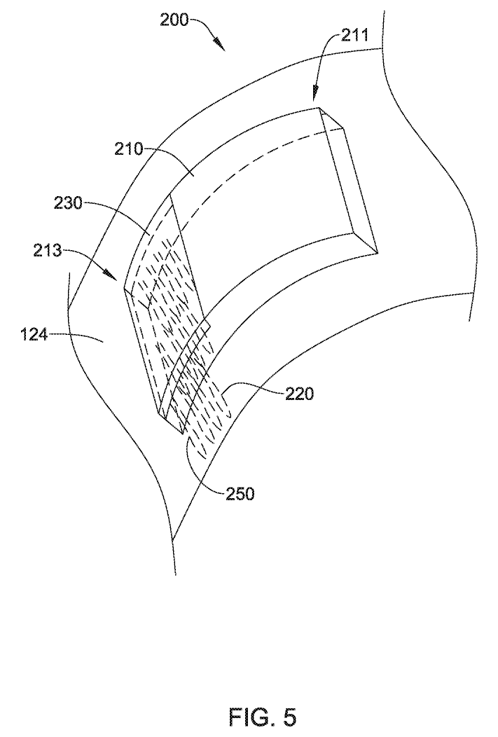

FIG. 4 is a schematic representation of the exemplary tendon repair implant 200 shown in FIG. 2A, showing the fibrous material of the second component 230 positioned only along the first, medial edge 211 of the tendon repair implant 200 and a portion of the upper surface of the first component 210 proximate the lateral edge 213 of the repair implant 200 exposed, and FIG. 5 is a schematic representation of the exemplary tendon repair implant 200 shown in FIG. 2A, showing the fibrous material of the second component 230 positioned only along the second, lateral edge 213 of the tendon repair implant 200 and a portion of the upper surface of the first component 210 proximate the medial edge 211 of the repair implant 200 exposed. In FIGS. 4 to 5, the repair implant 200 is shown with fibers 250 of the fibrous material of the second component 230 extending entirely through the thickness of the first component 210 and embedded into and/or entwined with the underlying tendon 124, such as with loops 220 of the fibers 250 embedded into the underlying tendon 124 for securement thereto.

As shown in FIG. 4, the second component 230 (e.g., layer) may be disposed on the first component 210 (e.g., layer) along the medial edge 211 of the repair implant 200. In some cases, the second component 230 may be pre-attached, pre-felted, glued, and in some cases, interwoven within the first component 210 along the medial edge 211 of the repair implant 200. In such an instance, the lateral edge 213 of the implant 200 may be secured to underlying bone and/or tissue with one or more tissue anchors (e.g., bone anchors and/or tendon anchors), if desired. As shown in FIG. 5, the second component 230 (e.g., layer) may be disposed on the first component 210 (e.g., layer) along the lateral edge 213 of the repair implant 200. In some cases, the second component 230 may be pre-attached, pre-felted, glued, and in some cases, interwoven within the first component 210 along the lateral edge 213 of the repair implant 200. In such an instance, the medial edge 211 of the implant 200 may be secured to underlying bone and/or tissue with one or more tissue anchors (e.g., bone anchors and/or tendon anchors), if desired. In some cases, the second component 230 may be two discrete, spaced apart strips, with a first segment pre- attached, pre-felted, glued, and in some cases, interwoven within the first component 210 along the medial edge 211 of the repair implant 200 and the second segment pre- attached, pre-felted, glued, and in some cases, interwoven within the first component 210 along the lateral edge 213 of the repair implant 200. Other arrangements of the second component 230 placed along a portion of the upper surface of the first component 210 are also contemplated.

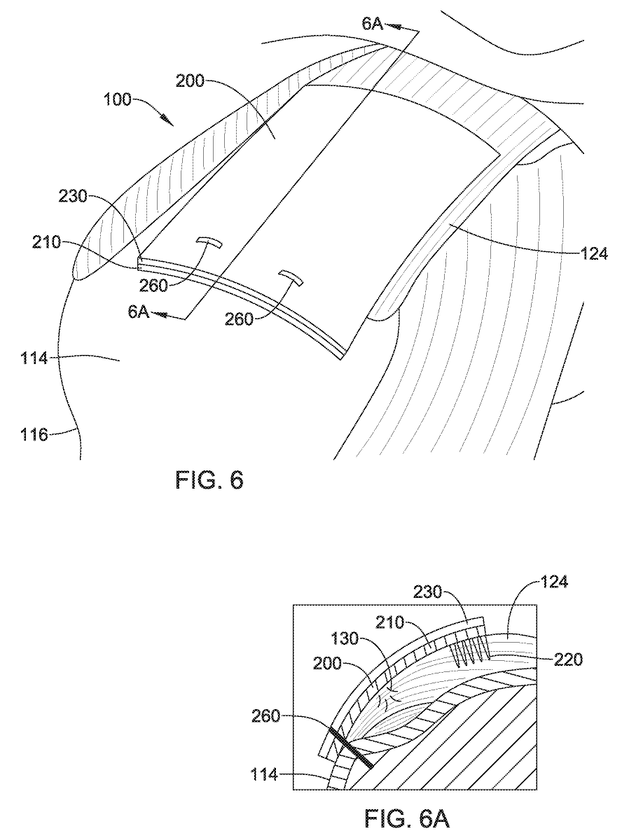

FIGS. 6 and 6A illustrate a method of securing the repair implant 200 to a damaged tissue site, e.g., tendon tear 130 (see FIG. 6A). The method may include placing the sheet-like implant 200 over the damaged tissue site. The sheet-like implant 200 may include a sheet-like structure having the first component 210 comprising a biological material, and the second component 230 comprising a fibrous felting material. In some cases, the second component 230 may be secured with the first component 210 as a single construct prior to placement over the tendon tear 130. For example, fibers of the second component 230 may be intertwined within the first component 210 prior to placement over the tendon tear 130. In other instances, the second component 230 may be placed over the previously placed first component 210 intraoperatively. The implant 200 may be secured over the damaged tissue by felting the second component 230 through the first component 210 to engage with the damaged tissue to affix the repair implant 200 to the tissue. In some cases, the fibers 250 of the second component 230 extend through the first component 210 and are embedded int the underlying tendon 124, and in some instances the fibers 250 form one or more loops 220 extending into the tendon tissue 124 configured to attach the implant 200 to the tissue. As shown in FIG. 6A, in some instances the medial edge of the implant 200 may be felted to underlying tendon tissue 124. In some cases, the sheet-like implant 200, such as the lateral edge of the implant 200 as shown, may be further affixed to the head 114 of the humerus 116 by one or more bone staples 260, or other similar and/or suitable bone anchors. The bone staple(s) 260, or other bone anchor(s), may extend through both the first component 210 and the second component 230 of the implant 200 and then into the underlying bone. By passing the bone staple(s) 260, or other bone anchor(s), through the second component 230, as well as the first component 210, the second component 230 may act as a reinforcing layer preventing the bone staple(s), or other one anchor(s) from tearing through the first component 210. In other words, the second component 230 may be stronger than the first component 210 such that the implant 200, including both the first component 210 and the second component 230, can withstand a greater loading force than the first component 210 alone.

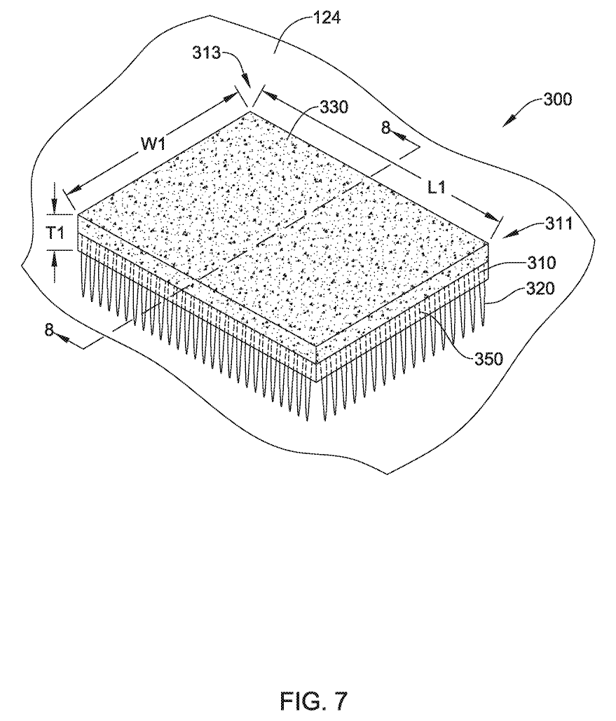

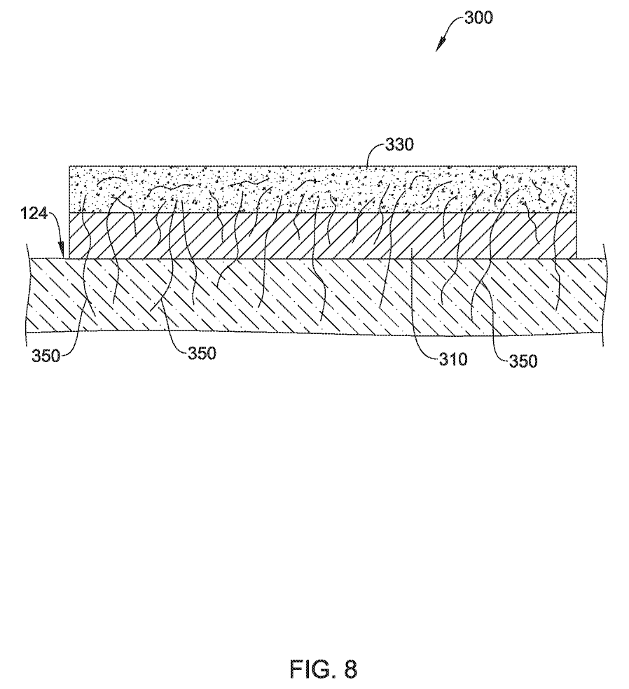

FIG. 7 is a schematic representation of an exemplary tendon repair implant 300 including a second, fibrous material layer 330 overlying a first, biological material layer 310. FIG. 8 is a schematic cross-sectional view of the exemplary tendon repair implant 300 shown in FIG. 7, taken at line 8-8. The repair implant 300 may be a multi-layer hybrid scaffold including a first or biological material layer 310 and a second or fibrous material layer 330 juxtaposed with the first layer 310. In some instances, the first layer 310 may be a collagen-based layer or other bioabsorbable layer, while the second layer 330 may be a felting layer including a plurality of feltable fibers. In some instances, the felt fibers of the second layer 330 may be biostable, non-bioabsorbable. In other instances, the felt fibers of the second layer 330 may be bioabsorbable. While the repair implant 300 is illustrated as having two discrete layers, it is contemplated that the repair implant 300 may include any number of layers desired, such as, but not limited to, one, two, three, four, or more. In some cases, the fibrous material layer 330 may be considered to be a structural layer, which may provide mechanical properties, such as strength, stiffness, resistance to creep, resistance to suture pull-out, etc., to support the load on the repair implant 300 upon initial implantation, while the biological material layer 310 (e.g., collagen) provides rapid tissue ingrowth. The repair implant 300 is defined by a longitudinal dimension Li (i.e., length), measured from a medial edge 311 to a lateral edge 313, a lateral dimension W1 (i.e., width), measured perpendicular to the longitudinal dimension Li, and a thickness T1, measured perpendicular to both the longitudinal dimension Li and the lateral dimension W1. The thickness Ti is measured from the upper surface to the lower surface of the implant 300. In some embodiments, lateral and longitudinal dimensions of the repair implant 300 may range from about 20 millimeters (mm) to 50 mm in the lateral direction W1 and 25 mm to 70 mm in the longitudinal direction L1. The thickness T1 of the sheet-like structure may be about 0.5 mm to 5 mm when dehydrated. It is contemplated that the thickness T1 of the implant 300 may be greater, in the range of about 1 mm to 10 mm, when hydrated. Upon implantation, the longitudinal dimension Li may extend generally in, or parallel to, the load bearing direction of the tendon.

Similar to that discussed with reference to FIGS. 2A to 6, the second layer 330 may be formed from a material that is amenable to a felting process. For example, the second layer 330 may be a resorbable felt or a non-resorbable felt including a plurality of felting fibers 350 or strands. In some cases, the second layer 330 includes a plurality of fibers 350 forming a fabric. In some cases, the second layer 330 may be formed from collagen, polyethylene, UHMWPE, PLA, PLLA, PGA, PLDL, PET, nylon, ABS, or any other suitable material. In some cases, the second layer 330, or the felting fibers 350 thereof, may include a color that differs from a color of the first layer 310. For example, the second layer 330 may be blue, green, yellow, red, or any other suitable color.

As shown in FIG. 8, some of the felting fibers 350 of the second layer 330 may extend into the first layer 310 without extending entirely through the thickness of the first layer 310, while others of the felting fibers 350 of the second layer 330 may extend entirely through the thickness of the first layer 310 and be embedded into the underlying soft tissue 124 to secure the implant 300 to the soft tissue 124.

In some embodiments, the tendon repair implant 300 may include a selected porosity and/or longitudinal pathways for tissue in-growth. In some useful embodiments, the sheet-like structure of the implant 300 comprises a material defining a plurality of pores that encourage tissue growth therein. In some cases, the first layer 310 may be a non-woven fibrous construct, such as a collagen-based scaffold. In some instances, the first layer 310 may be a collagen construct having longitudinally oriented collagen filaments. In other instances, the first layer 310 may be a woven construction including a weave pattern, for example. In some cases, a coating or substance that encourages tissue growth or in-growth may be applied to the surfaces of the sheet-like structure. The porosity and tissue in-growth allow for new collagen to integrate with collagen of the native tendon for functional load carrying. Further, the porosity of the first layer 310 of the implant 300 may be configured to allow fibers 350 of the second layer 330 to be felted therethrough. For example, when fibers 350 of the second layer 330 are felted through the thickness of the first layer 310 of the implant 300, the collagen fibers of the first layer 310 may be displaced to allow the fibers 350 of the second layer 330 to pass through the porosity of the first layer 310 to penetrate into underlying soft tissue 124. In some cases, the first layer 310 may include a density that allows a felting needle to easily push the felting material of the second layer 330 through the first layer 310 and into the tissue to attach the implant 300 to the tissue. In some cases, the implant 300 may be secured over the damaged tissue by felting the second layer 330 through the first layer 310 to engage with the damaged tissue to affix the repair implant 300 to the tissue. In some cases, fibers 350 of the second layer 330 extend through the plurality of pores of the first layer 310 between fibers of the first layer 310 and form one or more loops configured to attach the implant 300 to the tissue. In some cases, the fibers 350 may create their own pathway through the first layer 310 as defined by a needle, and thereby displace fibers of the first layer 310.

In some embodiments, the first layer 310 of the implant 300 has a pore size in the range of about 20 to about 400 microns. In some embodiments the pore size is in the range of about 100 microns to about 300 microns, and in some embodiments, the pore size is about 150 to about 200 microns. The porosity may be about 30% to about 90%, or it may be within the range of at least about 50% to about 85%, or in the range of at least about 70% to about 90%, in some instances. Examples of pore defining structures may include, but are not limited to open cell foam structures, mesh structures, micromachined layered structures, 3-D printed structures, and structures comprising a plurality of woven or non-woven fibers. In some embodiments, the fibers may be interlinked with one another. Various processes may be used to interlink the fibers with one another. Examples of processes that may be suitable in some applications include weaving, knitting, and braiding. In other instances, the fibers 350 may be randomly arranged within the first layer 310.

In some embodiments, the tendon repair implant 300 is bioresorbable, biodegradable or otherwise absorbable to provide transfer of additional load to native tendon over time. In some instances, by 2-3 months after implantation, the new tissue in-growth will have gained strength through remodeling such that more load may be transferred from the implant 300 to the new tissue and native tendon combination. Absorption of the implant 300 enables the new tissue, in combination with the native tendon, to carry all of the load and develop optimal collagen fiber alignment over time. Further, absorption avoids potential long-term problems with particles from non-absorbable materials. It is expected that tissue within the repair implant 300 will typically be developing and organizing during the first one to three months after implantation, so load sharing with the implant 300 is desired in some embodiments. After three months the tissue will typically be remodeling. As such, the mechanical properties of portions of the implant 300 may gradually decline to zero to enable the new tissue to be subjected to load without the implant 300 bearing any appreciable amount of the load.

The tendon repair implant 300 may comprise one or more bioabsorbable materials. Examples of bioabsorbable materials that may be suitable in some applications include those in the following list, which is not exhaustive: polylactide, poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen, poly(hydroxybutyrate ), polyanhydride, polyphosphoester; poly( amino acids), poly( alphahydroxy acid) or related copolymers materials.

The tendon repair implant 300 may be configured to allow loading and retention of biologic growth factors. The implant 300 and/or the growth factors may be configured to controllably release the growth factors. The implant 300 may be configured to allow transmission of body fluid to remove any degradation by- products in conjunction with a potential elution profile of biologics. The implant 300 may also include platelet rich plasma at the time of implantation or other biologic factor to promote healing and tissue formation.

In some instances, the tendon repair implant 300 may include multiple layers or surface coatings. As implanted, the bursal side of the implant 300 can include a layer or surface that will preferably slide against tissue without adherence. The tendon side of the implant 300 may include a layer or coating that is more compatible with fixation to the tendon surface.

In summary, the tendon repair implant or scaffold may include a bio- absorbable material. In some embodiments, the purpose of the scaffold is to protect an injured portion of a tendon during healing, provide a scaffold for new tissue growth, and/or temporarily share some of the tendon loads. The scaffold may induce additional tendon-like tissue formation, thereby adding strength and reducing pain, micro strains and inflammation.

Material(s) used in the implanted scaffold should be able to withstand the compression and shear loads consistent with accepted post-surgical shoulder motions. The perimeter of the scaffold may have different mechanical properties than the interior of the scaffold, such as for facilitating better retention of sutures, staples or other fastening mechanisms. The material(s) may be chosen to be compatible with visual, radiographic, magnetic, ultrasonic, or other common imaging techniques. The material(s) may be capable of absorbing and retaining growth factors with the possibility of hydrophilic coatings to promote retention of additives.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A tissue repair implant comprising:
a sheet-like scaffold having:
a first component comprising a biological material; and
a second component comprising a fibrous felt material formed of a plurality of fibers;
wherein fibers of the second component engage with the first component.

2. The tissue repair implant of claim 1, wherein the fibers of the second component extend entirely through a thickness of the first component to engage with a tissue to affix the tissue repair implant to the tissue.

3. The tissue repair implant of claim 1, wherein the fibers of the second component are glued to the first component.

4. The tissue repair implant of claim 1, wherein the fibers of the second component are pre-felted into the first component.

5. The tissue repair implant of claim 1, wherein biological material is a collagen layer and the fibrous felt material is a felt layer juxtaposed with an upper surface of the collagen layer.

6. The tissue repair implant of claim 1, wherein the second component extends across only a portion of an upper surface of the first component.

7. The tissue repair implant of claim 1, wherein the second component is secured to the first component only around an outer perimeter of the sheet-like scaffold.

8. The tissue repair implant of claim 1, wherein the fibers of the second component are intermingled within the first component via felting.

9. The tissue repair implant of claim 1, wherein fibers of the second component includes a color that differs from a color of the first component.

10. A tissue repair implant comprising:
a sheet-like scaffold having:
a first layer comprising an absorbable material; and
a second layer comprising a fibrous felt material formed of a plurality of fibers;
wherein the second layer is disposed along and juxtaposed with an upper surface of the first layer, and
wherein fibers of the felt material extend into the first layer to secure the second layer to the first layer.

11. The tissue repair implant of claim 10, wherein the absorbable material is collagen having collagen fibers.

12. The tissue repair implant of claim 11, wherein the fibers of the felt material are intertwined with collagen fibers.

13. The tissue repair implant of claim 12, wherein the felt material is a resorbable felt.

14. The tissue repair implant of claim 12, wherein the felt material is a non-resorbable felt.

15. The tissue repair implant of claim 10, wherein the fibers of the second layer include a color that differs from a color of the first layer.

16. The tissue repair implant of claim 10, wherein the fibers of the felt material extend entirely through a thickness of the first layer to engage with a tissue to affix the tissue repair implant to the tissue.

17. A method of securing a tissue repair implant to a damaged tissue, the method comprising:
placing a sheet-like implant over the damaged tissue, the sheet-like implant comprising:
a first component comprising a biological material and a second component comprising a fibrous felt material formed of a plurality of fibers; and
securing the sheet-like implant over the damaged tissue by felting the fibers of the second component through the first component to engage with the damaged tissue to affix the tissue repair implant to the tissue.

18. The method of claim 17, wherein the second component is attached to the first component prior to placing the sheet-like implant over the damaged tissue.

19. The method of claim 17, wherein the fibers of the second component are intermingled within the first component around an outer perimeter of the sheet-like implant.

20. The method of claim 17, wherein the step of placing the sheet-like implant over the damaged tissue includes:
placing the first component against the damaged tissue, and
thereafter, placing the second component against an upper surface of the first component only after the first component is placed against the damaged tissue.
